(19) United States
(12) Patent Application Publication
(10) Pub. No.: US 2023/0181663 A1
(43) Pub. Date: Jun. 15, 2023

(54) PREPARATION METHOD OF KAIXINSAN POWDER

(71) Applicants: Daiyin Peng, Hefei (CN); Chuanshan Jin, Hefei (CN); Can Peng, Hefei (CN); Peng Huang, Hefei (CN); Anhui University of Chinese Medicine, Hefei (CN)

(72) Inventors: Caiyun Zhang, Hefei (CN); Shujie Lv, Hefei (CN); Fangfang Gao, Hefei (CN); Xiaoxiao Shan, Hefei (CN); Junying Li, Hefei (CN); Xuan Yang, Hefei (CN); Yaoyao Xiao, Hefei (CN); Lanlan Ju, Hefei (CN); Shuangqing Yin, Hefei (CN)

(21) Appl. No.: 18/081,531

(22) Filed: Dec. 14, 2022

(30) Foreign Application Priority Data
Dec. 14, 2021 (CN) 202111525388.3

(51) Int. Cl. A61K 36/258; A61K 36/69; A61K 36/882; A61K 36/076; A61K 9/14

(52) U.S. Cl. CPC A61K 36/258; A61K 9/14; A61K 36/69; A61K 36/076; A61K 36/882; A61K 2236/15

(57) ABSTRACT

The present disclosure provides a preparation method of a Kaixinsan powder, including the following steps: pulverizing Radix Polygalae, Rhizoma Acori Graminei, ginseng, and Poria into a fine powder separately; and mixing each fine powder evenly in sequence according to a backing register method to obtain the Kaixinsan powder; where in the Kaixinsan powder, the fine powders each are required to pass through an 80-mesh sieve, and at least 95 wt% of each of the fine powders passes through a 100-mesh sieve. In the present disclosure, the fine powder of each medicinal material is capable of being mixed evenly, and has desirable dissolution effect and stability; and the Kaixinsan powder meets requirements for traditional Chinese medicine powders stipulated in Chinese Pharmacopoeia 2020 edition.

### CROSS REFERENCE TO RELATED APPLICATION

This patent application claims the benefit and priority of Chinese Patent Application No. 202111525388.3, filed with the China National Intellectual Property Administration on Dec. 14, 2021, the disclosure of which is incorporated by reference herein in its entirety as part of the present application.

### TECHNICAL FIELD

The present disclosure relates to the technical field of traditional Chinese medicines, in particular to a preparation method of a Kaixinsan powder.

### BACKGROUND

Powders refer to the dry powder preparations prepared by comminution and uniform mixing of drugs with suitable auxiliary materials. Powder is one of the oldest traditional formulations, and there are a lot of records about the powders in ancient “Treatise on Febrile Diseases”, “Supplementary Records of Famous Physicians”, and “Shen Nong’s Herbal Classic”.

Currently, powders are commonly used in traditional Chinese medicine formulations. Traditional Chinese medicine powder refers to a powdered preparation made of medicinal materials or medicinal material extracts by comminution and uniform mixing. Volume One of “Chinese Pharmacopoeia” (2015 edition) has collected more than 50 kinds of traditional Chinese medicine powders, such as a Qilisan Powder and a Bawei Qingxin Chenxiangsan Powder. In modem medicine, due to the development of modem solid dosage forms such as tablets and capsules, powders of chemicals are no longer common. Volume Two of “Chinese Pharmacopoeia” (2015 edition) only collects three kinds of powders of chemicals, such as a taurine powder and a fosfomycin tromethamine powder.

In addition to being directly used as a dosage form, the powder is also an intermediate for the preparation of other dosage forms including granules, capsules, tablets, suspensions, aerosols, dry powder inhalations, and sprays. Therefore, the preparation technology and requirements of powders are of general significance in other dosage forms.

Powders can be divided into oral powders and topical powders. The oral powder is generally dissolved or dispersed in water, diluents, or other liquids for taking, and can also be taken directly with water. The oral powder can exert a systemic therapeutic effect or local therapeutic effect. The powder should meet the following quality requirements during the production and storage: (1) The medicines for preparing the powder should be crushed into fine powder, where the oral powder should be a fine powder, and the topical powder should be an extremely-fine powder. (2) The powder should be dry, loose, evenly mixed, and consistent in color. (3) The powder should be stored in airtight areas, especially powders containing volatile or moisture-absorbing drugs should be stored in a completely airtight area. (4) Topical powders for burns or wounds should be sterile. (5) If a powder used for burn treatment is a non-sterile preparation, the powder should be marked with “non-sterile preparation” on the label and “for mild burns” in the product manual.

The classic prescription “Kaixinsan” powder was first recorded in “Important Prescriptions Worth a Thousand Gold for Emergency, Volume Fourteen, Small Intestine” written by Sun Simiao, a physician in the Tang Dynasty. It was recorded in the book that “the Kaixinsan powder, composed of four parts of Radix Polygalae, four parts of ginseng, one liang of Rhizoma Acori Graminei, and two liangs of Poria (that is, the weight ratio of the Radix Polygalae, the ginseng, the Rhizoma Acori Graminei, and the Poria is 1:1:1:2), is used for treating amnesia”. Throughout the whole prescription, the four herbs are mild-natured, and all of them have the effects of delighting and reinforcing intelligence, as well as calming the heart and tranquilizing the mind. The whole prescription is mainly based on supplementation, taking both symptoms and root causes into account, rigorous compatibility, and accurate medication. The prescription is simple but powerful. According to the basic theory of traditional Chinese medicine, the ginseng can greatly invigorate vitality, soothe the nerves and improve wisdom; the Poria can strengthen the spleen and calm the heart, and clear damp and promote diuresis; the Rhizoma Acori Graminei can resolve the phlegm, induce the resuscitation, and awaken the mind; and the Radix Polygalae can calm the heart and tranquilize the mind, and dispel the phlegm and induce the resuscitation. Moreover, the Radix Polygalae can be combined with the Rhizoma Acori Graminei to strengthen the effect of resolving phlegm and inducing resuscitation and can also be combined with the Poria to strengthen the coordinating between heart-yang and kidney-yin. Modem researches show that the “Kaixinsan” powder has a variety of pharmacological activities, such as anti-dementia, improving learning and memory, anti-oxidation, anti-depression, anti-aging, anti-fatigue, and promoting differentiation and regeneration of nerve cells. Accordingly, the powder has a significant curative effect on senile dementia, depression, anxiety, and other diseases.

In the original text of “Important Prescriptions Worth a Thousand Gold for Emergency”, a preparation method of the “Kaixinsan” powder is recorded as “the four herbs are ground and sieved ”, that is, the four kinds of medicinal materials are ground and then sieved through a fine sieve. At present, the “Kaixinsan” powder is generally prepared and used clinically in hospital pharmacies, and there is no mass-produced “Kaixinsan” powder on the market. However, the “Kaixinsan” prepared in hospitals has a relatively rough process, such that the four medicinal materials are mixed unevenly. As a result, when patients take the powder, the active ingredients may be different each time, and the dissolution rate may be inconsistent, thereby affecting the efficacy and therapeutic effect; moreover, there is a large amount of precipitation when taking the drug, resulting in poor patient compliance. In order to conduct inheritance, promotion, and application development of the classic prescription “Kaixinsan” powder, it is urgent to optimize the preparation process and improve the quality.

### SUMMARY

Based on the technical problems existing in the background, the present disclosure proposes a preparation method of a Kaixinsan powder. In the present disclosure, the fine powder of each medicinal material is capable of being mixed evenly, and has desirable dissolution effect and stability. The Kaixinsan powder meets all requirements for traditional Chinese medicine powders stipulated in Chinese Pharmacopoeia 2020 edition.

The present disclosure provides a preparation method of a Kaixinsan powder, including the following steps: pulverizing Radix Polygalae, Rhizoma Acori Graminei, ginseng, and Poria into a fine powder separately; and mixing each fine powder evenly in sequence according to a backing register method to obtain the Kaixinsan powder; wherein the Kaixinsan powder, the fine powders each are required to pass through an 80-mesh sieve, and at least 95 wt% of each of the fine powders passes through a 100-mesh sieve.

In the present disclosure, it is found that as the particle size decreases, the fine powders of medicinal materials each have a gradually increased angle of repose, decreased fluidity, and increased wettability. However, the fine powder also has increased hygroscopicity, which reduces the stability. The powder needs to maintain desirable dissolution effect and mixing effect, and a short mixing time. After pulverizing the Radix Polygalae, Rhizoma Acori Graminei, ginseng, and Poria into different particle sizes, the mixing state, mixing time, and dissolution rate and stability of active ingredients of “Kaixinsan” powder are investigated, thereby selecting an appropriate particle size.

Preferably, the fine powders of the Radix Polygalae, the Rhizoma Acori Graminei, the ginseng, and the Poria have a same particle size.

Preferably, the fine powders of the Radix Polygalae, the Rhizoma Acori Graminei, the ginseng, and the Poria each have a particle size of 80 mesh to 150 mesh.

Preferably, the fine powders of the Radix Polygalae, the Rhizoma Acori Graminei, the ginseng, and the Poria each have a particle size of 100 mesh to 120 mesh.

Preferably, during gradually mixing according to the backing register method, the Radix Polygalae, the Rhizoma Acori Graminei, the ginseng, and the Poria are added in sequence.

A process of gradually mixing by a backing register method includes the following steps: adding the Radix Polygalae, the Rhizoma Acori Graminei, the ginseng, and the Poria in sequence, and mixing through increment by equal quantity according to the formula of the Kaixinsan powder to obtain the Kaixinsan powder.

The Kaixinsan powder is mixed in a container; before the container is used, it is necessary to add an appropriate amount of the Poria in the container to saturate the container, and then export the Poria, followed by conducting gradually mixing according to the backing register method.

In the present disclosure, it is found that the preparation method and the order of adding medicinal materials have an influence on the mixing time as well as the hygroscopicity and stability of Kaixinsan powder. According to the order of Radix Polygalae, Rhizoma Acori Graminei, ginseng, and Poria, the mixing time can be shortened to save the preparation cycle, which is suitable for industrial production with saved energy consumption. In addition, in this order, the fine powders of Radix Polygalae, Rhizoma Acori Graminei, and ginseng can be wrapped by the fine powder of Poria with less hygroscopicity, thereby improving the stability of Kaixinsan powder.

Selecting an appropriate water content for the four medicinal materials can make the medicinal materials easier to crush and improve the stability.

Preferably, the Poria has a water content of less than or equal to 5.5 wt%.

Preferably, the Radix Polygalae has a water content of less than or equal to 7 wt%.

Preferably, the ginseng has a water content of less than or equal to 7 wt%.

Preferably, the Rhizoma Acori Graminei has a water content of less than or equal to 3 wt%.

Beneficial effects:

In the present disclosure, by designing the particle size and mixing method of the four medicinal materials, the Kaixinsan powder can be mixed uniformly in a relatively short time without particle stratification. The preparation method has shortened preparation period, saved energy consumption, and desirable dissolution effect and stability, which is suitable for industrial production. In addition, a large amount of precipitation is not produced when taking the Kaixinsan powder. The Kaixinsan powder meets requirements for traditional Chinese medicine powders stipulated in Chinese Pharmacopoeia 2020 edition.

### BRIEF DESCRIPTION OF THE DRAWINGS

FIG. 1 shows contents of dissolved substances in Kaixinsan powders prepared in Examples 1 to 5, where 80 mesh is Example 1, 100 mesh is Example 2, 120 mesh is Example 3, 150 mesh is Example 4, and 200 mesh is Example 5; and

FIG. 2 shows mixing states of Examples 6 to 9 when being mixed for 15 min, where a is Example 6, b is Example 7, c is Example 8, and d is Example 9.

### DETAILED DESCRIPTION OF THE EMBODIMENTS

The technical solutions of the present disclosure will be described in detail below with reference to specific examples.

### Examples 1 to 5

100 g of a Radix Polygalae coarse powder (in a particle size of 50 mesh) with a water content of 6 wt% to 6.5 wt% was divided into 5 parts of a same weight, and then pulverized into fine powders of 80 mesh, 100 mesh, 120 mesh, 150 mesh, and 200 mesh, respectively;

100 g of a ginseng coarse powder (in a particle size of 50 mesh) with a water content of 6 wt% to 6.5 wt% was divided into 5 parts of a same weight, and then pulverized into fine powders of 80 mesh, 100 mesh, 120 mesh, 150 mesh, and 200 mesh, respectively;

100 g of a Rhizoma Acori Graminei coarse powder (in a particle size of 50 mesh) with a water content of 2.5 wt% to 3 wt% was divided into 5 parts of a same weight, and then pulverized into fine powders of 80 mesh, 100 mesh, 120 mesh, 150 mesh, and 200 mesh, respectively;

200 g of a Poria coarse powder (in a particle size of 50 mesh) with a water content of 4 wt% to 5 wt% was divided into 5 parts of a same weight, and then pulverized into fine powders of 80 mesh, 100 mesh, 120 mesh, 150 mesh, and 200 mesh, respectively; and

According to a weight ratio of the Radix Polygalae, the ginseng, the Rhizoma Acori Graminei, and the Poria at 1:1:1:2, the fine powder of each medicinal material of each mesh was weighed separately, and then compounde d and mixed according to Table 1.

TABLE 1

Compounding situation

| | | |
|---|---|---|
| Groups | Mesh of medicinal material | Mixing method |
| Example 1 | 4 kinds of medicinal materials each are 80 mesh | Backing register method |
| Example 2 | 4 kinds of medicinal materials each are 100 mesh | |
| Example 3 | 4 kinds of medicinal materials each are 120 mesh | |
| Example 4 | 4 kinds of medicinal materials each are 150 mesh | |
| Example 5 | 4 kinds of medicinal materials each are 200 mesh | |

The mixing by backing register method specifically included: a small amount of light-colored and heavy Poria fine powder was added into a mortar to moisten and saturated the mortar, and the Poria fine powder was poured; a dark-colored and light Radix Polygalae fine powder was added into the mortar to make a base, a same volume of Rhizoma Acori Graminei fine powder was added, and then a mixed powder 1 was obtained by grinding and mixing; a ginseng fine powder with a same volume as the mixed powder 1 was added, and then ground and mixed to obtain a mixed powder 2; and the Poria fine powder with a same volume as the mixed powder 2 was added, and then ground and mixed to obtain the Kaixinsan powder.

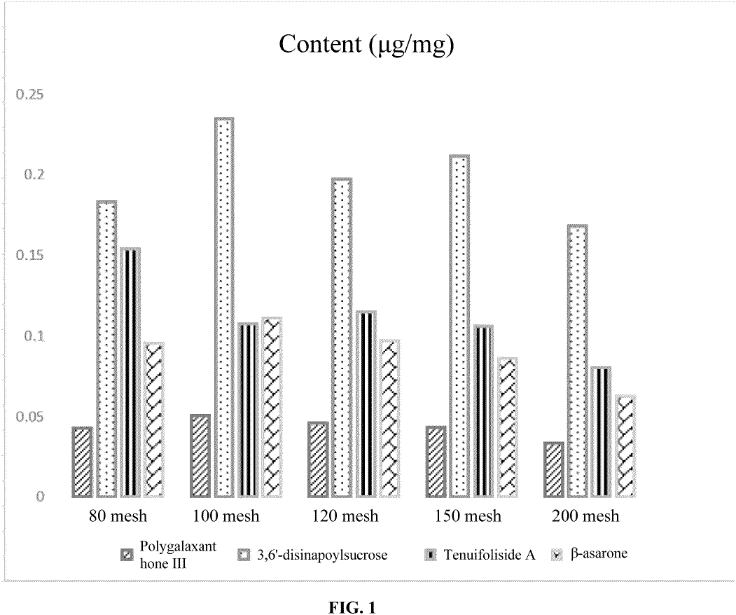

The Kaixinsan powders prepared in Examples 1 to 5 were tested, and the results were shown in Table 2 and FIG. 1.

A mixing standard was tested according to the appearance uniformity, specifically as follows: an appropriate amount of a test sample was placed on a smooth paper, tiled into about 5 cm², a surface of the test sample was flattened, and observed in a bright place. The test sample should have uniform color and luster, without patterns and color spots.

A moisture absorption rate detection method was as follows: a sample with a thickness of about 3 mm was placed on a bottom of a constant-weight open weighing bottle, and a weight of the sample was precisely weighed and recorded as Mₛₐₘₚₗₑ, and a total weight of the sample and the open weighing bottle was recorded as M₀; the open weighing bottle was placed in a closed environment with a relative humidity of 75%. After 2 d, a total weight of the sample and the open weighing bottle was accurately weighed and recorded as M₁, and the moisture absorption rate was calculated by: moisture absorption rate = (M₁-M₀)/Mₛₐₘₚₗₑ.

TABLE 2

Detection results

| Groups | Mixing time (min) | Mixing uniformity | Angle of repose (°) | Moisture absorption rate |
|---|---|---|---|---|
| Example 1 | 5 | Uniform | 34.1 | 6.9 |
| Example 2 | 10 | Uniform | 34.6 | 6.9 |
| Example 3 | 15 | Uniform | 34.9 | 7.0 |
| Example 4 | 25 | Uniform | 35.7 | 8.8 |
| Example 5 | 30 | Uniform | 37.4 | 9.5 |

It was seen from Table 2 that: when the particle size is 80 mesh to 120 mesh, the Kaixinsan powder had shorter mixing time, lower moisture absorption rate, and better stability.

The Kaixinsan powders prepared in Examples 1 to 5 were subjected to component detection, specifically as follows: 200 mg of the Kaixinsan powder was added to 4 ml of pure water, and allowed to stand in hot water at 80° C. to 100° C. for 10 min; 0.5 ml of a supernatant was added into chromatographic methanol until a methanol volume fraction was 75%, to obtain a solution to be tested; and contents of polygalaxanthone III, 3,6′-disinapoylsucrose, tenuifoliside A, and β-asarone in the Kaixinsan powder were detected by ultra-high liquid chromatography.

The chromatographic conditions of the ultra-high liquid chromatography were:
1) ultra-high liquid phase system: Thermo Fisher Ultimate 3000rs;
2) chromatographic column: ACQUITY UPLC BEHC18 column (2.1×100 mm, 1.7 µm);
3) a mobile phase A was acetonitrile, a mobile phase B was a 0.05% phosphoric acid aqueous solution, gradient elution was conducted;
4) the gradient elution procedure included:
   a. at 0 min to 9 min, the volume fraction of mobile phase A was 7% to 14%;
   b. at 9 min to 13 min, the volume fraction of mobile phase A gradually changed to 14% to 16%;
   c. at 13 min to 19 min, the volume fraction of mobile phase A gradually changed to 16% to 19%;
   d. at 19 min to 23 min, the volume fraction of mobile phase A gradually changed to 19% to 23%;
   e. at 23 min to 32 min, the volume fraction of mobile phase A gradually changed to 23% to 36%;
   f. at 32 min to 37 min, the volume fraction of mobile phase A gradually changed to 36% to 38%; and
   g. at 37 min to 41 min, the volume fraction of mobile phase A gradually changed to 38% to 42%;
5) after the gradient elution procedure was completed, at a volume fraction 7% of the mobile phase A, the gradient elution was continued for 10 min for next detection;
6) the column temperature was 30° C., the injection volume was 2 µL, the detection wavelength was 203 nm, and the flow rate was 0.2 mL/min.

The detection results were shown in FIG. 1. FIG. 1 shows contents of dissolved substances in Kaixinsan powders prepared in Examples 1 to 5, where 80 mesh is Example 1, 100 mesh is Example 2, 120 mesh is Example 3, 150 mesh is Example 4, and 200 mesh is Example 5.

As shown in FIG. 1: when the particle size was 100 mesh, the Kaixinsan powder had relatively high contents of the polygalaxanthone III, 3,6′-disinapoylsucrose, tenuifoliside A, and β-asarone; when the particle size was 200 mesh, the Kaixinsan powder has minimum contents of the polygalaxanthone III, 3,6′-disinapoylsucrose, tenuifoliside A, and β-asarone.

Taking into account comprehensively, the four medicinal materials each have a particle size of 80 mesh to 150 mesh, more preferably 100 mesh to 120 mesh.

### Examples 6 to 9

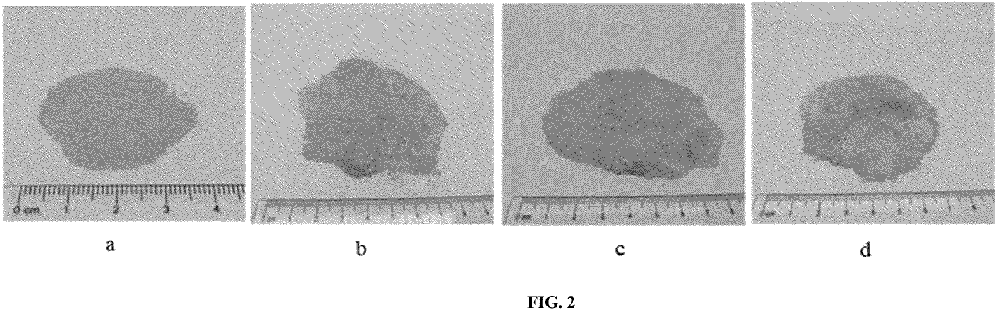

A 100-mesh Radix Polygalae fine powder, a 100-mesh ginseng fine powder, a 100-mesh Rhizoma Acori Graminei fine powder, and a 100-mesh Poria fine powder were mixed according to a weight ratio of 1:1:1:2 in different orders through increment by equal quantity, and the state when mixing for 15 min, time required for mixing, and moisture absorption rate after mixing well were counted. The results were shown in Table 3 and FIG. 2.

FIG. 2 shows mixing states of Examples 6 to 9 when being mixed for 15 min, where a is Example 6, b is Example 7, c is Example 8, and d is Example 9.

A mixing standard was tested according to the appearance uniformity, specifically as follows: an appropriate amount of a test sample was placed on a smooth paper, tiled into about 5 cm², a surface of the test sample was flattened, and observed in a bright place. The test sample should have uniform color and luster, without patterns and color spots.

A moisture absorption rate detection method was as follows: a sample with a thickness of about 3 mm was placed on a bottom of a constant-weight open weighing bottle, and a weight of the sample was precisely weighed and recorded as Mₛₐₘₚₗₑ, and a total weight of the sample and the open weighing bottle was recorded as M₀; the open weighing bottle was placed in a closed environment with a relative humidity of 75%. After 2 d, a total weight of the sample and the open weighing bottle was accurately weighed and recorded as M₁, and the moisture absorption rate was calculated by: moisture absorption rate = (M₁-M₀)/Mₛₐₘₚₗₑ.

TABLE 3

| Groups | Adding sequence | State when mixing for 15 min | Time required for mixing (min) | Moisture absorption rate after mixing well (%) |
|---|---|---|---|---|
| Example 6 | Radix Polygalae, Rhizoma Acori Graminei, ginseng, Poria | Uniform | 15 | 7.0 |
| Example 7 | Radix Polygalae, Rhizoma Acori Graminei, Poria, ginseng | Non-uniform | 20 | 7.3 |
| Example 8 | Radix Polygalae, Poria, Rhizoma Acori Graminei, ginseng | Non-uniform | 26 | 7.7 |
| Example 9 | Poria, Radix Polygalae, Rhizoma Acori Graminei, ginseng | Non-uniform | 26 | 7.8 |

As shown in Table 3 and FIG. 2, the mixing time was shortened according to the order of Radix Polygalae, Rhizoma Acori Graminei, ginseng, and Poria, with a lower moisture absorption rate.

The foregoing are merely descriptions of preferred specific embodiments of the present disclosure, but the protection scope of the present disclosure is not limited thereto. Any equivalent replacement or modification made within a technical scope of the present disclosure by a person skilled in the art according to the technical solutions of the present disclosure and inventive concepts thereof shall fall within the protection scope of the disclosure.

What is claimed is:

1. A preparation method of a Kaixinsan powder, comprising the following steps: pulverizing Radix Polygalae, Rhizoma Acori Graminei, ginseng, and Poria into a fine powder separately; and mixing each fine powder evenly in sequence according to a backing register method to obtain the Kaixinsan powder; wherein
in the Kaixinsan powder, the fine powders each are required to pass through an 80-mesh sieve, and at least 95 wt% of each of the fine powders passes through a 100-mesh sieve.

2. The preparation method of a Kaixinsan powder according to claim 1, wherein the fine powders of the Radix Polygalae, the Rhizoma Acori Graminei, the ginseng, and the Poria have a same particle size.

3. The preparation method of a Kaixinsan powder according to claim 1, wherein the fine powders of the Radix Polygalae, the Rhizoma Acori Graminei, the ginseng, and the Poria each have a particle size of 80 mesh to 150 mesh.

4. The preparation method of a Kaixinsan powder according to claim 2, wherein the fine powders of the Radix Polygalae, the Rhizoma Acori Graminei, the ginseng, and the Poria each have a particle size of 80 mesh to 150 mesh.

5. The preparation method of a Kaixinsan powder according to claim 1, wherein the fine powders of the Radix Polygalae, the Rhizoma Acori Graminei, the ginseng, and the Poria each have a particle size of 100 mesh to 120 mesh.

6. The preparation method of a Kaixinsan powder according to claim 2, wherein the fine powders of the Radix Polygalae, the Rhizoma Acori Graminei, the ginseng, and the Poria each have a particle size of 100 mesh to 120 mesh.

7. The preparation method of a Kaixinsan powder according to claim 3, wherein the fine powders of the Radix Polygalae, the Rhizoma Acori Graminei, the ginseng, and the Poria each have a particle size of 100 mesh to 120 mesh.

8. The preparation method of a Kaixinsan powder according to claim 4, wherein the fine powders of the Radix Polygalae, the Rhizoma Acori Graminei, the ginseng, and the Poria each have a particle size of 100 mesh to 120 mesh.

9. The preparation method of a Kaixinsan powder according to claim 1, wherein during gradually mixing according to the backing register method, the Radix Polygalae, the Rhizoma Acori Graminei, the ginseng, and the Poria are added in sequence.

10. The preparation method of a Kaixinsan powder according to claim 2, wherein during gradually mixing according to the backing register method, the Radix Polygalae, the Rhizoma Acori Graminei, the ginseng, and the Poria are added in sequence.

11. The preparation method of a Kaixinsan powder according to claim 3, wherein during gradually mixing according to the backing register method, the Radix Polygalae, the Rhizoma Acori Graminei, the ginseng, and the Poria are added in sequence.

12. The preparation method of a Kaixinsan powder according to claim 4, wherein during gradually mixing according to the backing register method, the Radix Polygalae, the Rhizoma Acori Graminei, the ginseng, and the Poria are added in sequence.

13. The preparation method of a Kaixinsan powder according to claim 5, wherein during gradually mixing according to the backing register method, the Radix Polygalae, the Rhizoma Acori Graminei, the ginseng, and the Poria are added in sequence.

14. The preparation method of a Kaixinsan powder according to claim 6, wherein during gradually mixing according to the backing register method, the Radix Polygalae, the Rhizoma Acori Graminei, the ginseng, and the Poria are added in sequence.

15. The preparation method of a Kaixinsan powder according to claim 7, wherein during gradually mixing according to the backing register method, the Radix Polygalae, the Rhizoma Acori Graminei, the ginseng, and the Poria are added in sequence.

16. The preparation method of a Kaixinsan powder according to claim 8, wherein during gradually mixing according to the backing register method, the Radix Polygalae, the Rhizoma Acori Graminei, the ginseng, and the Poria are added in sequence.

17. The preparation method of a Kaixinsan powder according to claim 1, wherein the water content of the Poria is less than or equal to 5.5 wt%.

18. The preparation method of a Kaixinsan powder according to claim 1, wherein the water content of the Radix Polygalae is less than or equal to 7 wt%.

19. The preparation method of a Kaixinsan powder according to claim 1, wherein the water content of the ginseng is less than or equal to 7 wt%.

20. The preparation method of a Kaixinsan powder according to claim 1, wherein the water content of the Rhizoma Acori Graminei is less than or equal to 3 wt%.
